(19) United States
(12) Patent Application Publication
(10) Pub. No.: US 2002/0165237 A1
(43) Pub. Date: Nov. 7, 2002

(54) Treatment of the insulin resistance syndrome

(76) Inventors: David Albert Fryburg, Groton, CT (US); Earl Michael Gibbs, Groton, CT (US); Nandan Parmanand Koppiker, Kent (GB)

Correspondence Address: Gregg C. Benson, Pfizer Inc., Patent Department, MS 4159, Eastern Point Road, Groton, CT, 06340, US

(21) Appl. No.: 09/927,525

(22) Filed: Aug. 10, 2001

Related U.S. Application Data
(60) Provisional application No. 60/224,928, filed on Aug. 11, 2000.
(60) Provisional application No. 60/256,431, filed on Dec. 18, 2000.
(60) Provisional application No. 60/266,083, filed on Feb. 2, 2001.
(60) Provisional application No. 60/292,506, filed on May 21, 2001.

(30) Foreign Application Priority Data
Dec. 15, 2000 (GB) 0030649.8
Mar. 15, 2001 (GB) 0106465.8
Mar. 15, 2001 (GB) 0106468.2
Jul. 13, 2001 (GB) 0117134.7

(51) Int. Cl. A61K 31/496

(52) U.S. Cl. 514/252.16

(57) ABSTRACT

Use of a selective cGMP PDE5 inhibitor or a pharmaceutical composition thereof in the preparation of a medicament for the curative, palliative or prophylactic treatment of the insulin resistance syndrome wherein the insulin resistance syndrome means the concomitant existence in a subject of two or more of: dyslipidemia; hypertension; type 2 diabetes mellitus, impaired glucose tolerance (IGT) or a family history of diabetes; hyperuricaemia and/or gout; a pro-coagulant state; atherosclerosis; or truncal obesity wherein said use can occur alone or in combination with other agents to treat the insulin resistance syndrome or individual aspects of the insulin resistance syndrome.

This invention relates to the use of selective cGMP PDE5 inhibitors and in particular to selective cGMP PDE5 inhibitor compounds such as the compound sildenafil for the treatment of the Insulin Resistance Syndrome.

The Insulin Resistance Syndrome as defined herein means the concomitant existence in a subject of two or more of: dyslipidemia, hypertension, type 2 diabetes mellitus or impaired glucose tolerance (IGT) or a family history of type 2 diabetes mellitus, hyperuricaemia and/or gout, a pro-coagulant state, atherosclerosis, truncal obesity. A family history of type 2 diabetes mellitus as defined herein means having a first degree relation, sibling, parent or grandparent with type 2 diabetes mellitus. At the centre of the Insulin Resistance Syndrome, also known as “Syndrome X” and “Metabolic Syndrome” in the biomedical literature is the common feature of tissue resistance to the action of insulin. This impaired biological response to insulin can be manifested in both the metabolic and vascular effects of insulin. Although there are monogenic syndromes of insulin resistance (IR), in which a definite gene has been identified as the cause of insulin resistance (such as leprechaunism), these are relatively rare. By contrast, the more common presentation of the IRS is associated with obesity (particularly abdominal) and appears to be polygenic.

The early adaptive response to insulin resistance in many individuals having the insulin resistance syndrome produces compensatory hyperinsulinaemia. As subjects with the insulin resistance syndrome become progressively insulin resistant, they manifest varying degrees of change in clinical parameters, including blood pressure, and/or increased levels of serum glucose, and/or cholesterol and/or triglycerides, and/or uric acid, and/or factors that increase coagulation. Once these clinical parameters have changed enough, the patient with the IRS may differentially manifest well-recognised clinical conditions or diagnoses. These conditions include:

1. Hypertension (high blood pressure);

2. Impaired Glucose Tolerance (IGT) or type 2 diabetes mellitus (DM);

3. Hyperlipidemia or dyslipidemia, particularly (but not limited to) hypertriglyceridemia;

4. Hyperuricemia or Gout;

5. Hypercoagulability (defined as an abnormal, increased tendency for clots to form, particularly inside blood vessels);

6. Atherosclerosis.

These clinical conditions are well-recognised risk factors for cardiovascular (coronary artery and cerebrovascular) disease.

It is difficult to estimate the prevalence of the Insulin Resistance Syndrome in the general populace due to both the diversity of the collective risk factors associated with the syndrome and the likelihood that many individuals affected by the Insulin Resistance Syndrome go undetected because they may exhibit no exterior symptoms and have no prior history of coronary heart disease. It is however possible to postulate that at a minimum the patient population at risk for the development of the Insulin Resistance Syndrome includes individuals with obesity, particularly truncal (abdominal) obesity. As obesity is an extremely common problem in the industrialized world and is associated with the clinical conditions mentioned above, it is very likely that the prevalence of IRS is very high. Considering this potential patient group alone forms an immense population potentially at risk for the development of complications of the Insulin Resistance Syndrome. For example in the United States in 1994, 23% of the population aged between 20 and 74 had hypertension, which accounted for 5 deaths per 100,000 population (1997). There will be an estimated 154,392,000 patients with diabetes world-wide in the year 2000. Of these, 15,000,000 will be in the US and 934,000 in the UK. The burden of disease for ischaemic heart disease for both sexes in the WHO region estimated for 1998 was 51,948,000 with a mortality of 7,375,000, constituting 13.7% of total mortality and ranking the highest in the mortality score. The burden of diabetes in both sexes in the WHO region estimated for 1998 was 11,668,000. Thus there exists a large medical need for an effective and safe oral therapy for the treatment of the Insulin Resistance Syndrome and prevention of the development of the Insulin Resistance Syndrome and its clinical consequences.

Resistance to the effects of insulin can also be observed in the diminished biological response of the endothelium to the vascular effects of insulin. That is, insulin promotes relaxation of blood vessel(s) at least in part through the action of nitric oxide. Nitric oxide generated in the endothelium then stimulates cGMP production in blood vessels and causes them to relax or dilate. This opening of the blood vessel allows more blood to flow, which is particularly important when more blood flow is needed to critical organs, like the heart. It has been demonstrated that there is a decreased release of nitric oxide (NO) from the endothelium of patients with insulin resistance. This decreased release of nitric oxide is not only from insulin, but also from other important vasodilators like acetylcholine. This so-called “endothelial dysfunction” contributes to the risk factors for cardiovascular disease which are associated with the Insulin Resistance Syndrome. It is thought that the vascular effect of insulin contributes to the effect of insulin to regulate metabolism, particularly, but not necessarily limited to, glucose metabolism.

In addition to the vascular actions of nitric oxide, NO also has direct effects on glucose uptake by skeletal muscle. That is, treatment with a NO-donor substance (nitroprusside) or an analogue of cGMP treatment in vitro increases glucose uptake (transport by GLUT4 glucose transporters). This vasodilation-independent pathway is described in G. J. Etgen, D. A. Fryburg and E. M. Gibbs in Diabetes, 46, 1997 pp. 1915-1919 the contents of which are incorporated herein by reference. It is proposed herein that, taken together, nitric oxide and cGMP likely have direct tissue level and vascular actions that influence, mediate, or mimic insulin's actions.

Further effects of impaired NO release by the endothelium include: an increase in vascular smooth muscle cell (VSMC) growth, proliferation and migration which are key steps in atherosclerotic plaque formation which can lead to stroke; an increase in platelet aggregation and adhesiveness (these effects on the platelet are also cGMP driven); an increase of lipid peroxidation and an effect on the inhibition of cell adhesion molecule expression including vascular cell adhesion molecule (VCAM-1), intracellular adhesion molecule (ICAM) and E-selectin. Impaired endothelial NO release also impacts on the activity of inflammatory cytokines such as tumour necrosis factor-α and the production of monocyte chemoattractant factor through decreased activity of the transcriptional activator nuclear factor k B.

There are examples in which the treatment of factors contributing to IRS (e.g., obesity) or the treatment of IRS itself, such as with troglitazone or Rezulin, improves many of these clinical conditions. For example, dieting alone or pharmacotherapeutic agents that induce weight loss will decrease blood pressure, blood glucose and triglycerides. Agents that are designed to improve insulin sensitivity can also favorably alter blood pressure, lipids, and blood glucose.

It is proposed herein that successful diagnosis and treatment of patients with the Insulin Resistance Syndrome (as defined above) with a selective PDE5 inhibitor, especially sildenafil, may lead to clinically relevant improvements in blood pressure and/or blood sugar and/or lipids and/or uric acid, and/or procoagulant factors. This treatment can occur alone or in combination with other therapeutics that improve the IRS. Improvement in these clinical conditions should reduce the risk of the development of cardiovascular disease in some of these patients as well as other complications of these individual disorders (including, but not limited to diabetic neuropathy, nephropathy, and retinopathy).

The present invention is concerned with the search for a pharmaceutical treatment for individuals with the Insulin Resistance Syndrome as defined hereinbefore.

Whilst the Insulin Resistance Syndrome has many manifestations an important underlying mechanistic basis for the condition resides in a resistance to both the vascular and metabolic effects of insulin. It is also understood that the underlying pathology of vascular resistance in insulin resistance syndrome, is a diminished amount of NO produced by the endothelial cells in response to insulin. In the insulin pathway in insulin resistant individuals, there may be impaired signalling of insulin for glucose uptake (from the phosphatidylinositol 3-kinase, PI3K, pathway) which may lead to an inefficient GLUT-4 transport mechanism. Whilst not wishing to be bound by any particular theory it is proposed herein that the pathway for the GLUT-4 transport mechanism and the cGMP-NO mechanistic pathway are somehow inter-linked.

For optimal functioning of the insulin signalling pathway for glucose uptake (via the GLUT-4 transport mechanism) it is preferable to have a normally functioning NO-cGMP pathway.

It is further proposed herein that amplification of the cGMP signal, using cGMP specific PDE5 inhibitors in patients with the Insulin Resistance Syndrome would help to optimise the insulin glucose uptake signal and improve insulin action at key tissues. By making tissues more sensitive to insulin, it is thereby also proposed herein that improvements in the clinical parameters of the IRS would result, including, but not limited to improvements in:

1. Blood glucose control: In patients with diabetes mellitus or impaired glucose tolerance (IGT), it is postulated herein that an improvement in insulin resistance should result in a decrease in plasma glucose concentrations (either fasting or after an oral glucose tolerance test or a meal). In a related manner, as regulated by the patient's pathophysiology, there will likely be an improvement in serum insulin concentrations in either the fasting state or after a glucose load or meal. These improvements in blood glucose control, should the subjects have type 2 diabetes mellitus, would manifest as improvements in measures of long-term blood glucose control, such as, but not limited to, haemoglobin A1c (glycosylated haemoglobin) or fructosamine; and/or

2. Blood pressure: It is postulated herein that an improvement in insulin resistance may also yield improvements in both systolic and diastolic blood pressure; and/or

3. Lipids: It is postulated herein that an improvement in insulin resistance may also yield improvements in serum lipids, including, but not limited to, serum cholesterol and triglycerides; and/or

4. Uric Acid: It is postulated herein that an improvement in insulin resistance may also yield improvements in serum uric acid; and/or

5. Coagulation Factors: It is postulated herein that an improvement in insulin resistance would also restore, towards normal, factors that worsen the procoagulant state.

These improvements in insulin resistance (improved sensitivity) may or may not be accompanied by improvement in compensatory hyperinsulinaemia and hence improve the components of the Insulin Resistance Syndrome.

cGMP PDE 5 inhibitors prevent the effect of the phosphodiesterase 5 enzyme that converts cGMP to inactive GMP thus increasing the amount of accumulated cGMP. This accumulation would amplify the vasodilatory, metabolic, and anti-atherogenic effects of the available nitric oxide and insulin. It is postulated herein that this amplification action (of plasma cGMP) will mitigate the adverse effects associated with the IRS and improve one or more of the associated conditions.

Sildenafil (Viagra®) is an orally-active, potent and selective inhibitor of cyclic guanosine monophosphate (cGMP) specific phosphodiesterase type 5 (PDE5) which is the predominant PDE5 isoenzyme in human corpora cavernosa. Consequently, sildenafil has been shown to be effective in the treatment of male erectile dysfunction. It is proposed herein that by inhibiting the cGMP to GMP conversion pathway, selective cGMP PDE5 inhibitors and in particular Sildenafil increase the intracellular concentrations of nitric oxide (NO) derived cGMP. This accumulation would amplify the vasodilatory, metabolic, and anti-atherogenic effects of the available nitric oxide and insulin.

Thus according to a first aspect of the present invention we provide a method of treating the insulin resistance syndrome which comprises treating the patient with an effective amount of a selective cGMP PDE5 inhibitor or a pharmaceutical composition thereof wherein the insulin resistance syndrome means the concomitant existence in a subject of two or more of: dyslipidemia; hypertension; type 2 diabetes mellitus, impaired glucose tolerance (IGT) or having a family history of diabetes; hyperuricaemia and/or gout; a pro-coagulant state; atherosclerosis; or truncal obesity.

Depending on the individual with IRS, the method may have a beneficial effect on one or more of conditions associated with the IRS as defined herein. Thus according to a further aspect the present invention additionally provides a method of treating type 2 diabetes mellitus or impaired glucose tolerance (IGT); or dyslipidemia, or hyperuricemia and/or gout, or the procoagulant state which comprises treating the patient with an effective amount of a cGMP PDE5 inhibitor or a pharmaceutical composition thereof. According to a yet further aspect the present invention additionally provides a method of treating type 2 diabetes mellitus or impaired glucose tolerance (IGT) in a patient wherein the patient does not have other risk factors associated with the IRS which comprises treating the patient with an effective amount of a selective pyrazolopyrimidinone cGMP PDE5 inhibitor as defined hereinafter or a pharmaceutically acceptable salt or a pharmaceutical composition thereof. According to a further aspect the present invention additionally provides a method for the prevention of progression in a subject of impaired glucose tolerance (IGT) to type 2 diabetes mellitus via treatment of a patient in need of such treatment with an effective amount of a selective pyrazolopyrimidinone cGMP PDE5 inhibitor as defined hereinafter or a pharmaceutically acceptable salt or a pharmaceutical composition thereof.

According to a second aspect the present invention provides a method of treating the insulin resistance syndrome which comprises treating the patient with an effective amount of a cGMP PDE5 inhibitor or a pharmaceutical composition thereof wherein the insulin resistance syndrome means the concomitant existence in a subject of three or more of: dyslipidemia; hypertension; type 2 diabetes mellitus, impaired glucose tolerance (IGT) or having a family history of diabetes; hyperuricaemia and/or gout; a pro-coagulant state; atherosclerosis; or truncal obesity.

According to a third aspect the present invention provides a method of treating the insulin resistance syndrome which comprises treating the patient with an effective amount of a cGMP PDE5 inhibitor or a pharmaceutical composition thereof wherein the insulin resistance syndrome means the concomitant existence in a subject of four or more of: dyslipidemia; hypertension; type 2 diabetes mellitus, impaired glucose tolerance (IGT) or having a family history of diabetes; hyperuricaemia and/or gout; a pro-coagulant state; atherosclerosis; or truncal obesity.

According to a fourth aspect the present invention provides a method of treating the insulin resistance syndrome which comprises treating the patient with an effective amount of a cGMP PDE5 inhibitor or a pharmaceutical composition thereof wherein the insulin resistance syndrome means the concomitant existence in a subject of five or more of: dyslipidemia; hypertension; type 2 diabetes mellitus, impaired glucose tolerance (IGT) or having a family history of diabetes; hyperuricaemia and/or gout; a pro-coagulant state; atherosclerosis; or truncal obesity.

According to a fifth aspect the present invention provides a method of treating the insulin resistance syndrome which comprises treating the patient with an effective amount of a cGMP PDE5 inhibitor or a pharmaceutical composition thereof wherein the insulin resistance syndrome means the concomitant existence in a subject of dyslipidemia; hypertension; type 2 diabetes mellitus or impaired glucose tolerance (IGT); and truncal obesity.

According to a sixth aspect the present invention provides a method of treating the insulin resistance syndrome which comprises treating a patient with type 2 diabetes mellitus, impaired glucose tolerance (IGT) or a family history of diabetes and at least one of the following conditions: dyslipidemia; hypertension; hyperuricaemia and/or gout; a pro-coagulant state; atherosclerosis; or truncal obesity with an effective amount of a cGMP PDE5 inhibitor or a pharmaceutical composition thereof.

According to a seventh aspect the present invention provides a method of treating the insulin resistance syndrome which comprises treating a patient with type 2 diabetes mellitus, impaired glucose tolerance (GT) or a family history of diabetes and at least two of the following conditions: dyslipidemia; hypertension; hyperuricaemia and/or gout; a pro-coagulant state; atherosclerosis; or truncal obesity with an effective amount of a cGMP PDE5 inhibitor or a pharmaceutical composition thereof.

According to an eighth aspect the present invention provides a method of treating the insulin resistance syndrome which comprises treating a patient with type 2 diabetes mellitus, impaired glucose tolerance (IGT) or a family history of diabetes and at least three of the following conditions: dyslipidemia; hypertension; hyperuricaemia and/or gout; a pro-coagulant state; atherosclerosis; or truncal obesity with an effective amount of a cGMP PDE5 inhibitor or a pharmaceutical composition thereof.

According to a ninth aspect the present invention provides a method of treating the insulin resistance syndrome which comprises treating a patient with type 2 diabetes mellitus, impaired glucose tolerance (IRS) or a family history of diabetes and at least three of the following conditions dyslipidemia; hypertension; hyperuricaemia and/or gout; a pro-coagulant state; atherosclerosis; or truncal obesity with an effective amount of a cGMP PDE5 inhibitor or a pharmaceutical composition thereof.

According to a tenth aspect the present invention provides a method of treating the insulin resistance syndrome which comprises treating a patient with type 2 diabetes mellitus, impaired glucose tolerance (IGT) or a family history of diabetes and at least four of the following conditions: dyslipidemia; hypertension; hyperuricaemia; a pro-coagulant state; atherosclerosis; or truncal obesity with an effective amount of a cGMP PDE5 inhibitor or a pharmaceutical composition thereof.

According to an eleventh aspect the present invention provides a method of treating the insulin resistance syndrome which comprises treating a patient with type 2 diabetes mellitus, impaired glucose tolerance (IGT) or a family history of diabetes and at least five of the following conditions: dyslipidemia; hypertension; hyperuricaemia and/or gout; a pro-coagulant state; atherosclerosis; or truncal obesity with an effective amount of a cGMP PDE5 inhibitor or a pharmaceutical composition thereof.

According to a further aspect the present invention additionally provides a method for the reduction in the risk of the development of cardiovascular disease in patients identified as being at risk of developing the insulin resistance syndrome wherein said method comprises treating the patient with an effective amount of a cGMP PDE5 inhibitor or a pharmaceutical composition thereof and wherein a patient at risk of developing the insulin resistance syndrome is defined as an individual having at least one of the following conditions: dyslipidemia; hypertension; type 2 diabetes mellitus, impaired glucose tolerance (IGT) or having a family history of diabetes; hyperuricaemia and/or gout; a pro-coagulant state; atherosclerosis; or truncal obesity and whom on subsequent analysis is found to have at least two of said conditions.

According to a further aspect the present invention provides a method for treating the insulin resistance syndrome as defined hereinbefore in a polygenic insulin resistant individual which comprises treating the individual with an effective amount of a cGMP PDE5 inhibitor or a pharmaceutical composition thereof.

Suitable PDE5i's for use in the pharmaceutical compositions according to the present invention are the cGMP PDE5i's hereinafter detailed. Particularly preferred for use herein are potent and selective cGMP PDE5i's.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124.

The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Preferred selective type V phosphodiesterase inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1 (R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1 R)-2-methoxy-1-methylethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

and pharmaceutically acceptable salts, polymorphs and solvates thereof.

Thus according to a preferred aspect the present invention provides for the use of a selective pyrazolopyrimidinone cGMP PDE5 inhibitor or a pharmaceutical composition thereof in the preparation of a medicament for the curative, palliative or prophylactic treatment of the insulin resistance syndrome in a mammal wherein the selective cGMP PDE5 inhibitor is selected from: sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one and 1-[[3-(3,4-dihydro-5-methyl-4-oxo-7-propylimidazolo[5,1-f]-as-trizin-2-yl)-4-ethoxyphenyl]sulphonyl]-4-ethylpiperzine or a pharmaceutically acceptable salt, solvate, pro-drug or polymorph thereof.

More particularly, the present invention provides a method of treating a patient with the insulin resistance syndrome which comprises treating the patient with an effective amount of a selective pyrazolopyrimidinone cGMP PDE5 inhibitor selected from sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or pharmaceutically acceptable salts, solvates, pro-drugs, polymorphs or pharmaceutical compositions thereof.

The invention also provides for the use of a selective pyrazolopyrimidinone cGMP PDE5 inhibitor and in particular sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one for the manufacture of a composition for the treatment or prophylaxis of the insulin resistance syndrome.

According to a further aspect the present invention additionally provides for the use of sildenafil or a pharmaceutically acceptable salt or of a pharmaceutical composition thereof for the treatment of the insulin resistance syndrome in a subject having type 2 diabetes mellitus, impaired glucose tolerance (IGT) or having a family history of diabetes and at least one of the following conditions: dyslipidemia; hypertension; hyperuricaemia and/or gout; a pro-coagulant state; atherosclerosis; or truncal obesity.

According to a yet further aspect the present invention additionally provides for the use of sildenafil or a pharmaceutically acceptable salt or of a pharmaceutical composition thereof for the treatment of the insulin resistance syndrome in a subject having type 2 diabetes mellitus, impaired glucose tolerance (IGT) or having a family history of diabetes and one or more of the following conditions dyslipidemia; hypertension; or truncal obesity.

According to a yet further still aspect the present invention additionally provides for the use of sildenafil or a pharmaceutically acceptable salt or of a pharmaceutical composition thereof for the treatment of the insulin resistance syndrome in a subject having type 2 diabetes mellitus, or impaired glucose tolerance (IGT) or having a family history of diabetes and dyslipidemia and hypertension and truncal obesity.

Preferred pharmaceutically acceptable salts of sildenafil for use herein are sildenafil citrate and sildenafil mesylate.

The cGMP PDE5 inhibitors as defined herein and in particular potent and selective cGMP PDE5 inhibitors and most particularly sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one are additionally useful for the manufacture of a composition for the treatment or prophylaxis of endothelial dysfunction.

Further type V PDE inhibitors suitable for use herein include:

(6R,12aR)-2,3,6,7,12,12a-hexahydro-2-methyl-6-(3,4-methylenedioxyphenyl) pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and

the compound of example 11 of published international application WO93/07124 (EISAI); and

compounds 3 and 14 from Rotella D P, J. Med. Chem., 2000, 43,1257.

Still other type cGMP PDE5 inhibitors useful in conjunction with the present invention include: 4-bromo-5-(pyridylmethylamino)-6-[3-(4-chlorophenyl)-propoxy]-3(2H)pyridazinone; 1-[4-[(1,3-benzodioxol-5- ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6- carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2- quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

It is to be understood that the contents of the above published patent applications, and in particular the general formulae of the therapeutically active compounds of the claims and the exemplified compounds therein are incorporated herein in their entirety by reference thereto.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC₅₀ against the PDE5 enzyme of less than 100 nanomolar (preferably, at less than 50 nanomolar).

According to a further aspect the present invention provides for the use of a selective pyrazolopyrimidinone cGMP PDE5 inhibitor or a pharmaceutical composition thereof in the preparation of a medicament for the curative, palliative or prophylactic treatment of type 2 diabetes mellitus or IGT in a mammal wherein the selective cGMP PDE5 inhibitor is selected from: sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one and 1-[[3-(3,4-dihydro-5-methyl-4-oxo-7-propylimidazolo[5,1-f]-as-trizin-2-yl)-4-ethoxyphenyl]sulphonyl]-4-ethylpiperzine or a pharmaceutically acceptable salt, solvate, pro-drug or polymorph thereof.

More particularly, the present invention provides a method of treating a patient with type 2 diabetes mellitus, IGT or IR which comprises treating the patient with an effective amount of a selective pyrazolopyrimidinone cGMP PDE5 inhibitor selected from sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or pharmaceutically acceptable salts, solvates, pro-drugs, polymorphs or pharmaceutical compositions thereof.

According to a further aspect still the invention also provides for the use of a selective pyrazolopyrimidinone cGMP PDE5 inhibitor and in particular sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one for the manufacture of a composition for the treatment or prophylaxis of type 2 diabetes mellitus, IGT or IR.

According to a further aspect the present invention additionally provides for the use of sildenafil or a pharmaceutically acceptable salt thereof or a pharmaceutical composition thereof for the treatment of type 2 diabetes mellitus, IGT or IR.

IC50 values for the cGMP PDE5 inhibitors may be determined using the PDE5 assay in the Test Methods Section hereinafter.

Preferably the cGMP PDE5 inhibitors used in the pharmaceutical compositions according to the present invention are selective for the PDE5 enzyme. Preferably they have a selectivity of PDE5 over PDE3 of greater than 100 more preferably greater than 300. More preferably the PDE5 has a selectivity over both PDE3 and PDE4 of greater than 100, more preferably greater than 300.

Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

According to a further aspect, the invention provides the use of a pharmaceutical medicament for use according to any aspect of the invention hereinbefore or hereinafter detailed which is adapted for administration by mouth, said medicament comprising a PDE5 inhibitor having and IC₅₀ of less than 100 nanomolar and a selectivity over PDE3 of greater than 100. Wherein said oral use is for the treatment of the insulin resistance syndrome, or type 2 diabetes mellitus or IGT or IR according to any aspect of the invention detailed herein before preferably said PDE5 inhibitor is a pyrazolopyrimidinone, more preferably sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one and 1-[[3-(3,4-dihydro-5-methyl-4-oxo-7-propylimidazolo[5,1-f]-as-trizin-2-yl)-4-ethoxyphenyl]sulphonyl]-4-ethylpiperzine or a pharmaceutically acceptable salt, solvate, pro-drug or polymorph thereof, and in particular sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one, and especially sildenafil.

Wherein said oral administration of a PDE5 inhibitor is for the treatment of insulin resistance; type 2 diabetes mellitus; impaired glucose tolerance (IGT); dislipidemia; hyperuricemia and/or gout; or a proco-agulant state then said PDE5 inhibitor is a pyrazolopyrimidinone, more preferably sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one and 1-[[3-(3,4-dihydro-5-methyl-4-oxo-7-propylimidazolo[5,1-f]-as-trizin-2-yl)-4-ethoxyphenyl]sulphonyl]-4-ethylpiperzine or a pharmaceutically acceptable salt, solvate, pro-drug or polymorph thereof, and in particular sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one, and especially sildenafil.

As proposed hereinbefore amplification of the cGMP signal, using cGMP specific PDE5 inhibitors in patients with the Insulin Resistance Syndrome would help to optimise the insulin glucose uptake signal and improve insulin action at key tissues. By making tissues more sensitive to insulin, it is thereby also proposed herein that improvements in the clinical parameters of the IRS would result. Thus, according to a further aspect the present invention additionally provides a method for improving insulin action (treating insulin resistance) wherein said method comprises treating a patient with the insulin resistance syndrome which comprises treating a patient with underlying insulin resistance with an effective amount of a selective pyrazolopyrimidinone cGMP PDE5 inhibitor (as detailed hereinbefore).

Thus the present invention additionally provides a method for treating insulin resistance which comprises treating a patient with underlying insulin resistance with an effective amount of a selective pyrazolopyrimidinone cGMP PDE5 inhibitor selected from sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or pharmaceutically acceptable salts, solvates, pro-drugs, polymorphs or pharmaceutical compositions thereof.

Depending on the individual with IRS, any of the methods detailed hereinbefore may have a beneficial effect on one or more of conditions associated with the IRS as defined herein. Thus according to a further aspect the present invention additionally provides a method of treating type 2 diabetes mellitus or impaired glucose tolerance (IGT); or dyslipidemia, or hyperuricemia, or the procoagulant state wherein said method comprises treating a patient with underlying insulin resistance with an effective amount of a selective pyrazolopyrimidinone cGMP PDE5 inhibitor (as detailed hereinbefore).

Thus the present invention additionally provides a method for treating type 2 diabetes mellitus or impaired glucose tolerance (IGT); or dyslipidemia, or hyperuricemia, or the procoagulant state which comprises treating a patient with an effective amount of a selective pyrazolopyrimidinone cGMP PDE5 inhibitor selected from sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or pharmaceutically acceptable salts, solvates, pro-drugs, polymorphs or pharmaceutical compositions thereof.

The pharmaceutically acceptable salts of the selective cGMP PDE5 inhibitor compounds as disclosed herein for use in the treatment of the insulin resistance syndrome in accordance with the present invention which contain a basic centre are, for example, non-toxic acid addition salts formed with inorganic acids such as hydrochloric, hydrobromic, hydroiodic, sulphuric and phosphoric acid, with carboxylic acids or with organo-sulphonic acids. Examples include the HCl, HBr, HI, sulphate or bisulphate, nitrate, phosphate or hydrogen phosphate, acetate, benzoate, succinate, saccarate, fumarate, maleate, lactate, citrate, tartrate, gluconate, camsylate, methanesulphonate, ethanesulphonate, benzene-sulphonate, p-toluenesulphonate and pamoate salts. The selective cGMP PDE5 inhibitor compounds for use in the present invention can also provide pharmaceutically acceptable metal salts, in particular non-toxic alkali and alkaline earth metal salts, with bases. Examples include the sodium, potassium, aluminium, calcium, magnesium, zinc and diethanolamine salts. For a review on suitable pharmaceutical salts see Berge et al, J. Pharm, Sci., 66, 1-19, 1977.

The cGMP PDE5i compounds suitable for use in accordance with any aspect of the present invention as disclosed herein, their pharmaceutically acceptable salts, and pharmaceutically acceptable solvates of either entity can be administered alone but, in human therapy will generally be administered in admixture with a suitable pharmaceutical excipient diluent or carrier selected with regard to the intended route of administration and standard pharmaceutical practice.

For example, the cGMP PDE5i compounds suitable for use in accordance with the present invention or salts or solvates thereof can be administered orally, buccally or sublingually in the form of tablets, capsules (including soft gel capsules), multi-particulate, gels, films, ovules, elixirs, solutions or suspensions, which may contain flavouring or colouring agents, for immediate-, delayed-, modified-, sustained-, dual-, controlled-release or pulsatile delivery applications. Such compounds may also be administered via fast dispersing or fast dissolving dosages forms or in the form of a high energy dispersion or as coated particles. Suitable pharmaceutical formulations may be in coated or un-coated form as desired.

Such solid pharmaceutical compositions, for example, tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules or HPMC capsules. Preferred excipients in this regard include lactose, starch, a cellulose, milk sugar or high molecular weight polyethylene glycols. For aqueous suspensions and/or elixirs, the cGMP PDE5i compounds may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The cGMP PDE5i compounds suitable for use in accordance with the present invention can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion or needle-free techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the selective cGMP PDE5 inhibitor compounds for use according to any aspect of the present invention as disclosed herein or salts or solvates thereof will usually be from 5 to 500 mg (in single or divided doses). For the treatment of the Insulin Resistance Syndrome the dosage may by via single dose, divided daily dose, multiple daily dose, acute dosing, continuous (chronic) daily dosing for a specified period which may be from one to five or 5 or more, such as up to 10 or more days. Alternatively the treatment of the Insulin Resistance Syndrome may be affected by continuous dosing, such as for example, via a controlled release dosage form wherein such continuous dosage form can be administered on a daily basis for a number of days or wherein such continuous dosing can be affected via a slow-release formulation which doses for more than one day at a time. For chronic conditions treatment may be effected via continuous daily dosing or via repeated regular dosing of controlled or sustained release formulation or such like. All references to the form of treatment may be equally applied to further aspects of the present invention as described herein such as for the treatment of type 2 diabetes mellitus, IGT or insulin resistance.

Thus, for example, tablets or capsules of the cGMP PDE5i compounds suitable for use in accordance with the present invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

### EXAMPLE TABLET FORMULATION

In general a tablet formulation could typically contain between about 0.01 mg and 500 mg of selective cGMP PDE5 inhibitor compounds for use in accordance with the present invention (or a salt thereof) whilst tablet fill weights may range from 50 mg to 1000 mg. An example formulation for a 10 mg tablet is illustrated:

Formulation 1

| Ingredient | % w/w |
|---|---|
| Sildenafil citrate | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity.

Formulation 2

A tablet is prepared using the following ingredients:

| | Quantity |
|---|---|
| | (mg/tablet) |
| sildenafil | 250 |
| cellulose, microcrystalline | 400 |
| silicon dioxide, fumed | 10 |
| stearic acid | 5 |
| total | 665 mg |

the components are blended and compressed to form tablets each weighing 665 mg.

Formulation 3

An intravenous formulation may be prepared as follows:

| | | |
|---|---|---|
| sildenafil | 100 | mg |
| isotonic saline | 1,000 | ml |

The cGMP PDE5i compounds suitable for use in accordance with the present invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark] or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The cGMP PDE5i compounds suitable for use in accordance with the present invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the cGMP PDE5i compounds suitable for use in accordance with the present invention or salts or solvates thereof can be administered in the form of a suppository or pessary, or they may be applied topically in the form of a gel, hydrogel, lotion, solution, cream, ointment or dusting powder. The cGMP PDE5i compounds suitable for use in accordance with the present invention or salts or solvates thereof may also be dermally or transdermally administered, for example, by the use of a skin patch. They may also be administered by the pulmonary or rectal routes.

The compounds may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the cGMP PDE5i compounds suitable for use in accordance with the present invention or salts or solvates thereof can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The cGMP PDE5i compounds suitable for use in accordance with the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration is the preferred route, being the most convenient. In circumstances where the recipient suffers from a swallowing disorder or from impairment of drug absorption after oral administration, the drug may be administered parenterally, sublingually or buccally.

For veterinary use, a compound, or a veterinarily acceptable salt thereof, or a veterinarily acceptable solvate or pro-drug thereof, is administered as a suitably acceptable formulation in accordance with normal veterinary practice and the veterinary surgeon will determine the dosing regimen and route of administration which will be most appropriate for a particular animal.

Thus, for example, tablets or capsules of selective cGMP PDE5 inhibitor compounds for use in accordance with the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

It is to be appreciated that all references herein to treatment include curative, palliative and prophylactic treatment and include acute treatment (taken as required) and chronic treatment (i.e. longer term continuous treatment).

The present invention additionally comprises treatment of the insulin resistance syndrome with a combination of a cGMP PDE₅ inhibitor compound as defined herein with one or more additional pharmaceutically active agents such as:

1) one or more naturally occurring or synthetic prostaglandins or esters thereof. Suitable prostaglandins for use herein include compounds such as alprostadil, prostaglandin E₁, prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate; and/or

2) one or more α-adrenergic receptor antagonist compounds, α-blockers. Suitable compounds for use herein include: the α-adrenergic receptor blockers as described in PCT application WO99/30697 published on Jun. 14, 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine (α₂-blocker), rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cardiotonic agent such as pirxamine; and/or

3) one or more NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginine, ginseng, zizphi fructus, molsidomine, Re-2047, nitrosylated maxisylyte derivatives such as NMI-678-11 and NMI-937 as described in published PCT application WO 0012075; and/or

4) one or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂; and/or

5) one or more dopaminergic agents, preferably apomorphine or a selective D2, D3 or D2/D₃agonist such as, pramipexole and ropirinol (as claimed in WO-0023056), L-Dopa or carbidopa, PNU95666 (as claimed in WO-0040226); and/or

6) one or more vasodilator agents. Suitable vasodilator agents for use herein include nimodepine, pinacidil, cyclandelate, isoxsuprine, chloroprumazine, halo peridol, Rec 15/2739, trazodone, and/or

7) one or more thromboxane A2 agonists; and/or

8) one or more ergot alkoloids; Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride; and/or

9) one or more compounds which modulate the action of natruretic factors in particular atrial naturetic factor (also known as atrial naturetic peptide), B type and C type naturetic factors such as inhibitors of neutral endopeptidase; and/or

10) one or more angiotensin receptor antagonists such as losartan; and/or

11) one or more substrates for NO-synthase, such as L-arginine; and/or

12) one or more calcium channel blockers such as amlodipine; and/or

13) one or more antagonists of endothelin receptors and inhibitors or endothelin-converting enzyme; and/or

14) one or more cholesterol lowering agents such as statins (e.g. atorvastatin/Lipitor-trade mark) and fibrates; and/or

15) one or more antiplatelet and antithrombotic agents, e.g. tPA, uPA, warfarin, hirudin and other thrombin inhibitors, heparin, thromboplastin activating factor inhibitors; and/or

16) one or more insulin sensitising agents such as Rezulin, Avandia or Actos and hypoglycaemic agents such as, but not limited to, glipizide (sulfonylureas), metformin, or acarbose; and/or

17) one or more acetylcholinesterase inhibitors such as donezipil; and/or

18) one or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

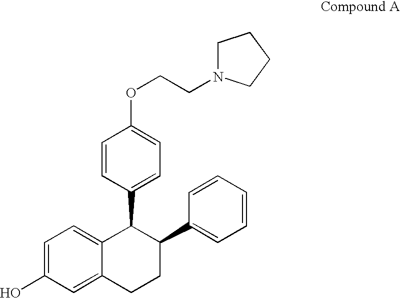

23) one or more of a further PDE inhibitor, more particularly a PDE 2, 4, 7 or 8 inhibitor, preferably a PDE2 inhibitor said inhibitors preferably having an IC50 against the respective enzyme of less than 100 nM: and/or

24) one or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM, more preferably less than 50 nM, suitable NPY and in particular NPY1 inhibitor compounds are described in EP-A-1097718; and/or

25) one or more of vasoactive intestinal peptide (VIP), VIP mimetic, more particularly mediated by one or more of the VIP receptor subtypes VPAC1, VPAC or PACAP (pituitary adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil); and/or

26) one or more of a melanocortin receptor agonist or modulator or melanocortin enhancer, such as melanotan 11, PT-14, PT-141 or compounds claimed in WO-09964002, WO-00074679, WO-09955679, WO-00105401, WO-00058361, WO-00114879, WO-00113112, WO-09954358 and/or

27) one or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993; and/or

28) one or more of a testosterone replacement agent (inc dehydroandrostendione), testosternone (Tostrelle), dihydrotestosterone or a testosterone implant; and/or

29) one or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone); and/or

30) one or more of a modulator of transporters for noradrenaline, dopamine and/or serotonin, such as bupropion, GW-320659

31) one or more of a purinergic receptor agonist and/or modulator; and/or

32) one or more of a neurokinin (NK) receptor antagonist, including those described in WO-09964008; and/or

33) one or more of an opioid receptor agonist, antagonist or modulator, preferably agonists for the ORL-1 receptor and/or;

34) one or more of an agonist or modulator for oxytocin/vasopressin receptors, preferably a selective oxytocin agonist or modulator and/or;

35) one or more modulators of cannabinoid receptors.

36) one or more CNS active agents; and/or

37) one or more compounds which inhibit angiotensin-converting enzyme such as enapril, and one or more combined inhibitors of angiotensin-converting enzyme and neutral endopeptidase such as omapatrilat; and/or

38) L-DOPA or carbidopa; and/or

39) one or more steroidal or non-steroidal anti-inflammatory agents; and/or

40) one or more protein kinase C-β inhibitors such as LY333531; and/or

41) one or more activators of AMP-activated protein kinase such as 5-amino-4-imidazolecarboxamide ribonucleoside; and/or

42) insulin; and/or

43) weight loss agents such as sibutramine or orlistat; and/or

44) one or more dipeptidyl peptidase IV inhibitors such as NVP DPP728 or P32/98; and/or

45) one or more glucagon antagonists such as NNC25-2504

46) one or more agents that inhibit PTP1B such as PTP112; and/or

47) one or more agents that reduce PTP1B levels using antisense technology; and/or

48) one or more glycogen synthase kinase-3 inhibitors such as Chir98014; and/or

49) one or more GLP-1 agonists such as GLP1, NN-2211 or exendin 4; and/or

50) one or more PPAR-gamma agonists such as Rezulin, Avandia, Actos or CS011; and/or,

51) one or more PPAR-alpha agonists such as fenofibrate; and/or

52) one or more dual PPAR-alpha/PPAR-gamma agonists such as farglitazar, rosiglitasone, pioglitazone, GW1929, DRF2725, AZ242 or KRP 297, and/or

53) one or more sorbitol dehydrogenase inhibitors such as CP-470711; and/or

54) one or more aldose reductase inhibitors such as zopoirestat, zenarestat, or fidarestat.

55) one or more preparations of growth hormone or growth hormone secretagogues; and/or

56) one or more of an NEP inhibitor, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719 and/or;

According to a still further aspect the present invention comprises treatment of type 2 diabetes mellitus or IGT or IR with a combination of a cGMP PDE5 inhibitor compound which is a pyrazolopyrimidinone, more preferably sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one, 1-[[3-(3,4-dihydro-5-methyl-4-oxo-7-propylimidazolo[5,1-f]-as-trizin-2-yl)-4-ethoxyphenyl]sulphonyl]-4-ethylpiperzine or a pharmaceutically acceptable salt, solvate, pro-drug or polymorph thereof, and in particular sildenafil, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one or 5-[2-ethoxy-5-(4-ethylpiperazin-1-ylsulphonyl)pyridin-3-yl]-3-ethyl-1-(2-methoxyethyl)-1,6-dihydro-7H-pyrazolo[4,3-d]pyrimidin-7-one, and especially sildenafil with one or more additional pharmaceutically active agents wherein said additional agent comprises one or more agent from (1) to (56) as defined herein before.

Preferred combinations for use according to any of the aspects of the present invention as described herein include a combination of a cGMP PDE5i, and in particular sildenafil with one or more additional agents selected from (16), (40), (41), (42), (43), (50), (51), (52), (53) or (54) as detailed hereinbefore.

Pharmacokinetics

Bioavailability

Preferably, the PDE5 inhibitor compounds for use in the present invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity. Dissolution, the solubilisation of the drug by the aqueous contents of the gastro-intestinal tract (GIT), can be predicted from in vitro solubility experiments conducted at appropriate pH to mimic the GIT. Preferably the PDE5 inhibitor compounds for use according to the present invention have a minimum solubility of 50 mcg/ml. Solubility can be determined by standard procedures known in the art such as described in Adv. Drug Deliv. Rev. 23, 3-25,1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +3. The log D can be determined by standard procedures known in the art such as described in J. Pharm. Pharmacol. 1990, 42:144.

Cell monolayer assays such as Caco-2 add substantially to prediction of favourable membrane permeability in the presence of efflux transporters such as p-glycoprotein, so-called Caco-2 flux. Preferably, compounds for use according to the present invention have a Caco-2 flux of greater than 2×10⁻⁶ cms⁻¹, more preferably greater than 5×10⁻⁶ cms⁻¹. The Caco-2 flux value can be determined by standard procedures known in the art such as described in J. Pharm. Sci, 1990, 79, 595-600.

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stablity in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28,1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226. and as described in J. Pharm. Sci 79, 7, p595-600 (1990), and Pharm. Res. vol 14, no. 6 (1997).

PDE5 inhibitor—Test Methods

Phosphodiesterase (PDE) Inhibitory Activity

Preferred PDE compounds suitable for use in accordance with the present invention are potent and selective cGMP PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) can be obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜⅓ Km) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

Functional Activity

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppi.), abstract 153P).

In vivo Activity

Compounds were screened in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

Effect of Specific Selective PDE5 inhibitors on Insulin Resistance Syndrome in animals—Effects on Plasma Glucose and Serum Triglyceride Levels in ob/ob Mice

Biological Data

Experimental Protocol

Test Compounds:

The selective PDE5 inhibitor compounds to be tested were solubilized in 10% DMSO/0.1% pluronics and dosed via oral gavage using mouse oral feeding needles (20 gauge, Popper & Sons, Inc., New Hyde Park, N.Y.). A volume of 4 ml/kg weight was administered for each dose. Compounds were tested at doses ranging from 1-50 mg/kg. Alternatively, the test selective PDE5 inhibitor compound was administered in the drinking water and found to produce similar reductions in plasma glucose and triglycerides to the reductions observed for the same compound when administered by oral gavage.

Experimental Animals:

Male ob/ob mice obtained from Jackson Laboratories (Bar Harbor, ME) were used in the studies at 6 to 10 weeks of age. Mice were housed five per cage and allowed free access to D11 mouse chow (Purina, Brentwood, Mo.) and water.

Experimental Protocol:

Mice were allowed to acclimate to the Pfizer animal facilities for 1 week prior to the start of the study. On day one, retro-orbital blood samples were obtained and plasma glucose was determined as described hereinafter. Mice were then sorted into groups of five such that mean plasma glucose concentrations for each group did not differ. On day one, mice were dosed with vehicle or a test selective PDE5 inhibitor compound only in the afternoon. Subsequently, mice were dosed twice a day on day 2-4 in the morning and in the afternoon. On day 5, the mice received an a.m. dose and bled 3 hours later for plasma preparation for glucose and triglyceride analysis as described below. Alternatively, test selective PDE5 inhibitor compound was administered in the drinking water commencing on the afternoon of day 1 and continuing through day 5, when mice were then bled for plasma preparation for glucose and triglyceride analysis as described below. Terminal plasma samples were collected on day 5 following the retro-orbital sinus bleed as described below. Body weight was measured on days 1 and 5 of the study, and food consumption was assessed over the 5 day period.

Terminal Bleed and Tissue Collection:

On the morning of the last day of the study mice were dosed with test compound or vehicle at approximately 8:00 am. Three hours after dosing, 25 μL of blood was obtained via the retro-orbital sinus and added to 100 μL of 0.025% heparinized-saline in Denville Scientific microtubes. The tubes were spun at the highest setting in a Beckman Microfuge 12 for 2 minutes. Plasma was collected for plasma glucose and triglyceride determination. The mice were then sacrificed by decapitation and ˜1 ml of blood was collected in Becton-Dickinson Microtainer brand plasma separator tubes with lithium heparin. The tubes were spun in a Beckman Microfuge 12 at the maximum setting for five minutes. Plasma was collected in 1.5 ml Eppendorf tubes and snap frozen in liquid nitrogen. Plasma samples were stored at −80° C. until analyzed.

Metabolite and Hormone Analysis:

Plasma glucose and triglycerides were measured using the Alcyon Clinical Chemistry Analyzer (Abbott Laboratories, Abbott Park, Ill.) using kits supplied by Abbott. Plasma cGMP was measured using the Biotrak enzyme-immunoassay system by Amersham (Piscataway, N.J.). Via a similar technique the plasma insulin can be assessed by the Mercodia ELISA Insulin kit by ALPCO (Uppsala, Sweden). All assays were conducted according to instructions provided by the manufacturers.

Statistical Analysis:

Comparisons between drug treatments and appropriate vehicles were done by Student's t-test.

Results (Summary):

Selective PDE5 inhibitors have been demonstrated to reduce the plasma glucose and serum triglyceride levels produced by ob/ob mice in accordance with the biological test methods detailed hereinbefore.

Results

Table 1 illustrates the changes in plasma glucose levels over a 5 day period observed with selective PDE5 inhibitor compounds.

Selective PDE5 Compound A: 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one

Selective PDE5 Compound B: 5-(2-ethoxy-5-morpholinoacetylphenyl)-1-methyl-3-n-propyl-1,6-dihydro-7H-pyrazolo[4,3-d]pyrimidin-7-one

TABLE 1

| | Changes in Plasma Glucose |
|---|---|
| | Concentrations (mg/dl) |
| Vehicle | −9 ± 22 |
| PDE5 A − 10 mg/kg | −115 ± 34* |
| PDE5 A − 50 mg/kg | −105 ± 25* |
| PDE5 B − 25 mg/kg | −97 ± 32* |

The data in Table 1 are presented as mean ±standard error of the mean. These numbers reflect absolute decreases in plasma glucose levels. Significant differences from the Vehicle control are indicated as *p<0.05.

Table 2 illustrates the change in plasma cGMP and plasma triglyceride levels in ob/ob mice observed with the test selective PDE5 inhibitor compounds A and B.

TABLE 2

| | Plasma cGMP | Plasma Triglyceride |
|---|---|---|
| | Level (mg/dl) | Level (mg/dl) |
| Vehicle | 9.8 ± 0.5 | 178 ± 16 |
| PDE5 A − 10 mg/kg | 48.3 ± 19.0\hat{ } | 163 ± 10 |
| PDE5 B − 25 mg/kg | 30.7 ± 3.3** | 143 ± 7\hat{ } |

The data in Table 2 are presented as mean ±standard error of the mean. Significant differences from the Vehicle control as indicated as ˆ p<0.1, *p<0.05, **p<0.01.

Table 3 illustrates the reduction in plasma glucose levels over a 5 day period observed with selective a PDE5 inhibitor compound administered in the drinking water of the mice.

Selective PDE5 Compound C: sildenafil

TABLE 3

| | Changes in Plasma Glucose |
|---|---|
| | Concentrations (mg/dl) |
| Vehicle | 25 ± 25 |
| PDE5 C − 9 mg/kg | −27 ± 34 |
| PDE5 C − 22 mg/kg | −15 ± 27 |
| PDE5 C − 45 mg/kg | −36 ± 22\hat{ } |

The data in Table 3 are presented as mean ±standard error of the mean. Positive values in this table reflect a decrease in plasma glucose level. Significant differences from the Vehicle control are indicated as ˆ p<0.1.

Table 4 illustrates the triglyceride levels in ob/ob mice treated with the test selective PDE5 inhibitor compound C administered in the drinking water of the mice.

TABLE 4

| | Plasma Triglyceride |
|---|---|
| | Level (mg/dl) |
| Vehicle | 204 ± 13 |
| PDE5 C − 9 mg/kg | 163 ± 14* |
| PDE5 C − 22 mg/kg | 212 ± 20 |
| PDE5 C − 45 mg/kg | 151 ±10** |

The data in Table 4 are presented as mean ±standard error of the mean. Significant differences from the Vehicle control as indicated as *p<0.05, **p<0.01.

Taken together, these experimental results in the hyperglycemic, insulin-resistant ob/ob mouse suggest that selective PDE5 inhibition improves metabolic parameters associated with IRS.

Further these results suggest that treatment with selective PDE5 inhibitors can result in decreases in plasma glucose concentrations. As detailed herein before decreases in plasma glucose concentrations are consistent with an improvement in insulin resistance which is a clinical parameter of the IRS, and, as further detailed hereinbefore such improvements, in subjects with type 2 diabetes mellitus would manifest as improvements in for example haemoglobin A1c.

These results also suggest that treatment with selective PDE5 inhibitors can result in improvements in serum lipid levels. As detailed herein before an improvement in serum lipid levels (such as in triglyceride levels) is consistent with an improvement in insulin resistance which is a clinical parameter of the IRS. Such improvements, in subjects with the IRS (as defined herein) would manifest as improvements in for example dyslipidemia (hypertriglyceridaemia).

These results in the hyperglycemic, insulin-resistant ob/ob mouse additionally suggest that continuous treatment with a selective PDE5 inhibitor can improve metabolic parameters associated with IRS in 5 days or less.

Clinical Trial Data 1

In a 28-day clinical trial with sildenafil, serum triglyceride levels were obtained before and at the end of dosing from non-diabetic subjects. Doses included in the study were 10 mg, 25 mg and 50 gm of sildenafil or placebo, dosed once daily. These subjects had the insulin resistance syndrome, as defined by the risk factors as defined hereinbefore. Data are presented in tables 5 and 6 below to illustrate that subjects had evidence of the insulin resistance syndrome.

Table 5 illustrates the sub-division of the IRS components amongst the subjects studied.

Coronary artery disease (CAD)/ischaemic heart disease (1HD) is one clinical end-point in subjects having the Insulin Resistance Syndrome. For this reason the presence of CAD/IHD was taken as evidence to support the existence of the Insulin Resistance Syndrome in patients with only one of the defined risk factors.

TABLE 5

| IRS Component | Placebo | Sildenafil | Total |
|---|---|---|---|
| Dyslipidemia (Hypertriglyceridaemia) | 40 | 74 | 114 |
| Obesity (BMI > 26) | 26 | 47 | 73 |
| Hypertension | 10 | 12 | 22 |
| Coronary artery disease (h/o angina, | 8 | 5 | 13 |
| arrhythmia, MI, i.e., atherosclerosis) | | | |
| Hyperuricaemia | 0 | 2 | 02 |

BMI-Body Mass Index

Table 6 illustrates the collective totals of the number of IRS components within the subject group.

TABLE 6

| | | |
|---|---|---|
| 1 component | 114 | T |
| 2 component | 80 | 73 (T + O); 4 (T + H); 3 (T + I) |
| 3 component | 24 | 6 (T + O + I); 14 (T + O + H); |
| | | 2 (T + I + H); 2 (T + O + U) |
| 4 component | 4 | T + O + I + H |

T = Raised Triglycerides (Dyslipidemia), O = Obesity, H = Hypertension, I = CAD/IHD, U = Hyperuricaemia

Preliminary data are presented below demonstrating reductions in serum triglyceride levels with sildenafil treatment. Falls in serum triclyceride levels (in mg/dl) of 100.3; 67.3; and 23.9 were observed for the 10, 25 and 50 mg sildenafil groups respectively, compared to 19.9 mg/dl fall in the placebo group. This represents a fall of about 40%; 31% and 12% fall on treatment with sildenafil from baseline values (mg/dl) of 255, 213 and 191 compared to 10.7% from a baseline of 185 mg/dl for placebo.

The reduction of serum triglycerides that was seen in the sildenafil group was statistically significantly different compared to placebo (p=0.0457). There was also a trend seen in HDL (p=0.0539). These changes, in non-diabetic subjects who have many features of the IRS, are consistent with improvements in the insulin resistance syndrome.

Further these results are consistent with improvements in insulin resistance in non-diabetic subjects who have many features of the IRS when treated with sildenafil. Thus according to a further aspect the present invention additionally comprises treatment of insulin resistance with a selective pyrazolopyrimdinone cGMP PDE5 inhibitor (as defined hereinbefore).

Clinical Trial Data 2

Adult subjects with Diabetes Mellitus were treated chronically with sildenafil citrate in an out-patient, multicentre study. Subjects were taking several different glucose-lowering agents (including, but not limited to, metformin, insulin, or sulfonylureas) or were treated with diet alone. Glycosylated haemoglobin (HbA1c) was determined prior to treatment and at the end of the study. Glycosylated haemoglobin (HbA1_{c}) is a recognised measure of chronic glucose control. In this study a significant improvement in glucose control was observed in said subjects with type 2 diabetes mellitus when treated with sildenafil citrate (Viagra™). These significant improvements were consistently observed across the subject group irrespective of their background therapy.

These results are consistent with improvements in the IRS in adult subjects when treated with sildenafil. Further these results are consistent with improvements in glucose control in patients having type 2 diabetes mellitus when treated with sildenafil. Thus the present invention provides a method of treatment of type 2 diabetes mellitus comprising treating a subject in need of such treatment with a selective pyrazolopyrimidinone cGMP PDE5 inhibitor (as defined hereinbefore) and especially sildenafil. In particular said treatment is effected by the oral route.

The results of clinical trial 2 are supporting for the use of a selective cGMP PDE5 inhibitor as defined hereinbefore, and especially sildenafil for the treatment of IGT.

Additionally these results are consistent with improvements in the IRS in adult subjects when treated with a combination of sildenafil and glucose-lowering agents. Thus the present invention additionally comprises the combination of a selective PDE5 inhibitor and a glucose-lowering agent for the treatment of the IRS. In particular said combined treatment is effected by the oral route.

According to a further aspect the present invention provides a combination therapy suitable for use in the treatment of the IRS, IR, type 2 diabetes mellitus, impaired glucose tolerance (IGT), hyperuricaemia and/or gout, dyslipidemia, the procoagulant state or truncal obesity wherein said combination comprises a selective cGMP PDE5 inhibitor, preferably a pyrazolopyrimidinone, especially sildenafil with an additional agent active as defined hereinbefore and preferably (16), (40), (41), (42), (50), (52), (53) and/or (54), more preferably one or more of: weight loss agents, sulfonyl ureas, insulin, Rezulin, Avandia, Actos, Glipizide, Metformin, Acarbose, rosiglitasone, pioglitazone, farglitazar, LY333531, CS011, PPAR-alpha agonists, and/or CP-470711.

According to a yet further aspect said combination treatment(s) is/are effected orally and further may be in the form of a kit.

Taken together, the results from both the animal and human trials are consistent with improvements in the IRS as well as in clinical parameters associated with the IRS. That is, improvements in triglycerides in both diabetic and non-diabetic experiments as well as improvement in glucose in those with diabetes support the activity of PDE5 inhibitor compounds, including but not limited to sildenafil, on the IRS.

1. A method of treating the insulin resistance syndrome in a mammal comprising administering to said mammal an effective amount of a selective cGMP PDE5 inhibitor or a pharmaceutically acceptable salt, solvate or composition thereof, wherein the insulin resistance syndrome means the concomitant existence in said mammal of two or more of: dyslipidemia; hypertension; type 2 diabetes mellitus, impaired glucose tolerance (IGT) or a family history of diabetes; hyperuricaemia and/or gout; a pro-coagulant state; atherosclerosis; or truncal obesity.

2. A method of claim 1 wherein said mammal has type 2 diabetes mellitus, impaired glucose tolerance (IGT) or a family history of diabetes and at least one or more of: dyslipidemia; hypertension; hyperuricaemia and/or gout; a pro-coagulant state; atherosclerosis; or truncal obesity.

3. A method of claim 2 wherein said selective cGMP PDE5 inhibitor is sildenafil or sildenafil citrate.

4. A method of claim 1 wherein said mammal has type 2 diabetes mellitus, impaired glucose tolerance (IGT) or a family history of diabetes and at least two or more of: dyslipidemia; hypertension; hyperuricaemia and/or gout; a pro-coagulant state; atherosclerosis; or truncal obesity.

5. A method of claim 1 wherein said mammal has type 2 diabetes mellitus, impaired glucose tolerance or a family history of diabetes and at least three or more of: dyslipidemia; hypertension; hyperuricaemia and/or gout; a pro-coagulant state; atherosclerosis; or truncal obesity.

6. A method of claim 1 wherein said mammal has type 2 diabetes mellitus, impaired glucose tolerance or a family history of diabetes and at least four or more of: dyslipidemia; hypertension; hyperuricaemia and/or gout; a pro-coagulant state; atherosclerosis; or truncal obesity.

7. A method of claim 1 wherein said mammal has type 2 diabetes mellitus, impaired glucose tolerance or a family history of diabetes and at least five or more of: dyslipidemia; hypertension; hyperuricaemia and/or gout; a pro-coagulant state; atherosclerosis; or truncal obesity.

8. A method of claim 1 wherein said mammal has type 2 diabetes mellitus or impaired glucose tolerance; dyslipidemia; hypertension; and truncal obesity.

9. A method of claim 1 wherein said mammal has three or more of: type 2 diabetes mellitus, impaired glucose tolerance (IGT) or a family history of diabetes; dyslipidemia; hypertension; hyperuricaemia and/or gout; a pro-coagulant state; atherosclerosis; or truncal obesity.

10. A method of claim 1 wherein said mammal has four or more of: type 2 diabetes mellitus, impaired glucose tolerance (IGT) or a family history of diabetes; dyslipidemia; hypertension; hyperuricaemia and/or gout; a pro-coagulant state; atherosclerosis; or truncal obesity.

11. A method of claim 1 wherein said mammal has five or more of: type 2 diabetes mellitus, impaired glucose tolerance (IGT) or a family history of diabetes; dyslipidemia; hypertension; hyperuricaemia and/or gout; a pro-coagulant state; atherosclerosis; or truncal obesity.

12. A method of claim 1 wherein said mammal has dyslipidemia, hypertension, type 2 diabetes mellitus or impaired glucose tolerance (IGT) and truncal obesity.

13. A method of claim 1 wherein the selective cGMP PDE5 inhibitor is sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, (6R, 12aR)-2,3,6,7,12,12a-hexahydro-2-methyl-6-(3,4-methylenedioxyphenyl) pyrazino[2′,1′:6,1]pyrido[3,4-b]indole-1,4-dione; 2-[2-ethoxy-5-(4-ethyl-piperazin-1-yl-1-sulphonyl)-phenyl]-5-methyl-7-propyl-3H-imidazo[5, 1-f][1,2,4]triazin-4-one or 1-[[3-(3,4-dihydro-5-methyl-4-oxo-7-propylimidazolo[5,1-f]-as-triazin-2-yl)-4-ethoxyphenyl]sulphonyl]-4-ethylpiperazine or a pharmaceutically acceptable salt, solvate, pro-drug, polymorph or pharmaceutical composition thereof.

14. A method of claim 13 wherein the selective cGMP PDE5 inhibitor is sildenafil, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

15. A method of claim 14 wherein the selective cGMP PDE5 inhibitor is sildenafil citrate.

16. A method of claim 1 wherein said mammal has: type 2 diabetes mellitus, impaired glucose tolerance (IGT) or a family history of diabetes; dyslipidemia;
hypertension; and truncal obesity.

17. A method of treating the insulin resistance syndrome in a mammal comprising administering to said mammal an effective amount of a selective cGMP PDE5 inhibitor or a pharmaceutically acceptable salt, solvate or pharmaceutical composition thereof.

18. A method of claim 17 comprising daily dosing, wherein said dosing is in the form of single, multiple or divided doses.

19. A method of claim 18 comprising daily dosing for 5 or more days, wherein said daily dosing is in the form of single, multiple or divided doses.

20. A method of claim 17 comprising continuous dosing for 5 or more days, wherein said continuous dosing is in the form of single or multiple continuous release doses.

21. A method of treating the insulin resistance syndrome in a mammal comprising administering to said mammal an effective amount of a selective cGMP PDE5 inhibitor in combination with one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents.

22. A method of claim 21 wherein said selective cGMP PDE5 inhibitor is sildenafil, sildenafil citrate or a pharmaceutical composition thereof and wherein said mammal has: type 2 diabetes mellitus, impaired glucose tolerance (IGT) or a family history of diabetes; dyslipidemia; hypertension; and truncal obesity.

23. A method of treating type 2 diabetes mellitus in a mammal comprising administering to said mammal an effective amount of a selective pyrazolopyrimidinone cGMP PDE5 inhibitor, a pharmaceutically acceptable salt thereof or a pharmaceutical composition thereof.

24. A method of claim 23 wherein the selective pyrazolopyrimidinone cGMP PDE5 inhibitor is sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one, or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, or a pharmaceutically acceptable salt, solvate, pro-drug, polymorph or a pharmaceutical composition thereof.

25. A method of claim 24 wherein the selective pyrazolopyrimidinone cGMP PDE5 inhibitor is sildenafil, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

26. A method of claim 24 wherein the selective cGMP PDE5 inhibitor is sildenafil or a pharmaceutically acceptable salt, solvate or pharmaceutical composition thereof.

27. A method of claim 26 wherein the selective cGMP PDE5 inhibitor is sildenafil citrate.

28. A method of claim 26 comprising daily dosing, wherein said dosing is in the form of single, multiple or divided doses.

29. A method of claim 28 comprising daily dosing for 5 or more days, wherein said daily dosing is in the form of single, multiple or divided doses.

30. A method of claim 26 comprising continuous dosing for 5 or more days, wherein said continuous dosing is in the form of single or multiple continuous release doses.

31. A method of treating type 2 diabetes mellitus in a mammal comprising administering to said mammal an effective amount of sildenafil citrate in combination with one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors one or more insulin sensitising agents; and one or more hypoglycaemic agents.

32. A method of treating impaired glucose tolerance (IGT) in a mammal comprising administering to said mammal an effective amount of a selective pyrazolopyrimidinone cGMP PDE5 inhibitor or a pharmaceutical composition thereof.

33. A method of claim 32 wherein the selective pyrazolopyrimidinone cGMP PDE5 inhibitor is sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one, or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, or a pharmaceutically acceptable salt, solvate, pro-drug, polymorph or a pharmaceutical composition thereof.

34. A method of claim 33 wherein the selective pyrazolopyrimidinone cGMP PDE5 inhibitor is sildenafil, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

35. A method of claim 33 wherein said selective cGMP PDE5 inhibitor is sildenafil, a pharmaceutically acceptable salt thereof or a pharmaceutical composition thereof.

36. A method of claim 35 wherein the selective pyrazolopyrimidinone cGMP PDE5 inhibitor is sildenafil citrate.

37. A method of claim 36 comprising daily dosing, wherein said dosing is in the form of single, multiple or divided doses.

38. A method of claim 37 comprising daily dosing for 5 or more days, wherein said daily dosing is in the form of single, multiple or divided doses.

39. A method of claim 36 comprising continuous dosing for 5 or more days, wherein said continuous dosing is in the form of single or multiple continuous release doses.

40. A method of treating of impaired glucose tolerance (IGT) in a mammal comprising administering to said mammal an effective amount of sildenafil citrate in combination with one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents.

41. A method of treating insulin resistance (IR) in a mammal comprising administering to said mammal an effective amount of a selective pyrazolopyrimidinone cGMP PDE5 inhibitor or a pharmaceutical composition thereof.

42. A method of claim 41 wherein the selective pyrazolopyrimidinone cGMP PDE5 inhibitor is sildenafil, 5-(2-ethoxy-5-morpholinoacetylphenyl)-1-ethyl-3-n-propyl-1,6-dihydro-7H-pyrazolo[4,3-d]pyrimidin-7-one, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one, or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, or a pharmaceutically acceptable salt, solvate, pro-drug, polymorph or a pharmaceutical composition thereof.

43. A method of claim 42 wherein the selective pyrazolopyrimidinone cGMP PDE5 inhibitor is sildenafil, 5-[2-ethoxy-5-(4-ethylpiperazin-1-ylsulphonyl)pyridin-3-yl]-3-ethyl-2-(2-methoxyethyl)-2,6-dihydro-7H-pyrazolo[4,3-d]pyrimidin-7-one or 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

44. A method of claim 42 wherein said selective cGMP PDE5 inhibitor is sildenafil, a pharmaceutically acceptable salt thereof or a pharmaceutical composition thereof.

45. A method of claim 44 wherein the selective pyrazolopyrimidinone cGMP PDE5 inhibitor is sildenafil citrate.

46. A method of claim 44 comprising daily dosing, wherein said dosing is in the form of single, multiple or divided doses.

47. A method of claim 46 comprising daily dosing for 5 or more days, wherein said daily dosing is in the form of single, multiple or divided doses.

48. A method of claim 44 comprising continuous dosing for 5 or more days, wherein said continuous dosing is in the form of single or multiple continuous release doses.

49. A method of treating of insulin resistance (IR) in a mammal comprising administering to said mammal an effective amount of sildenafil citrate in combination with one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents.

50. A method of claim 21 wherein said selective cGMP PDE5 inhibitor is sildenafil citrate and said further components are selected from one or more of: weight loss agents, sulfonyl ureas, insulin, Rezulin, Avandia, Actos, Glipizide, Metformin, Acarbose, rosiglitasone, pioglitazone, farglitazar, LY333531, CS011, PPAR-alpha agonists, and CP-470711.

51. A method of claim 1 wherein said mammal is a polygenic mammal.

52. A method of claim 31 wherein said selective cGMP PDE5 inhibitor is sildenafil citrate and said further components are selected from one or more of: weight loss agents, sulfonyl ureas, insulin, Rezulin, Avandia, Actos, Glipizide, Metformin, Acarbose, rosiglitasone, pioglitazone, farglitazar, LY333531, CS011, PPAR-alpha agonists, and CP-470711.

53. A method of claim 40 wherein said selective cGMP PDE5 inhibitor is sildenafil citrate and said further components are selected from one or more of: weight loss agents, sulfonyl ureas, insulin, Rezulin, Avandia, Actos, Glipizide, Metformin, Acarbose, rosiglitasone, pioglitazone, farglitazar, LY333531, CS011, PPAR-alpha agonists, and CP-470711.

54. A method of claim 49 wherein said selective cGMP PDE5 inhibitor is sildenafil citrate and said further components are selected from one or more of: weight loss agents, sulfonyl ureas, insulin, Rezulin, Avandia, Actos, Glipizide, Metformin, Acarbose, rosiglitasone, pioglitazone, farglitazar, LY333531, CS011, PPAR-alpha agonists, and CP-470711.

55. A method of claim 1 comprising administering said cGMP PDE5 inhibitor orally.

56. A method of claim 21 comprising administering said cGMP PDE5 inhibitor orally.

57. A method of claim 23 comprising administering said cGMP PDE5 inhibitor orally.

58. A method of claim 32 comprising administering said cGMP PDE5 inhibitor orally.

59. A method of claim 41 comprising administering said cGMP PDE5 inhibitor orally.

60. A method of claim 1 wherein said cGMP PDE5 inhibitor has an IC₅₀ against PDE5 of less than 100 nM and a selectivity ratio of PDE5 over PDE3 of more than 100.

61. A method of claim 21 wherein said cGMP PDE5 inhibitor has an IC₅₀ against PDE5 of less than 100 nM and a selectivity ratio of PDE5 over PDE3 of more than 100.

62. A method of claim 23 wherein said cGMP PDE5 inhibitor has an C₅₀ against PDE5 of less than 100 nM and a selectivity ratio of PDE5 over PDE3 of more than 100.

63. A method of claim 32 wherein said cGMP PDE5 inhibitor has an IC₅₀ against PDE5 of less than 100 nM and a selectivity ratio of PDE5 over PDE3 of more than 100.

64. A method of claim 41 wherein said cGMP PDE5 inhibitor has an IC₅₀ against PDE5 of less than 100 nM and a selectivity ratio of PDE5 over PDE3 of more than 100.

65. A kit containing a treatment for the insulin resistance syndrome, wherein the insulin resistance syndrome means the concomitant existence in said mammal of two or more of: dyslipidemia; hypertension; type 2 diabetes mellitus, impaired glucose tolerance (IGT) or a family history of diabetes;
hyperuricaemia and/or gout; a pro-coagulant state; atherosclerosis; or truncal obesity comprising:
a) a therapeutically effective amount of a selective pyrazolopyrimidinone cGMP PDE5 inhibitor and a pharmaceutically acceptable carrier in a first unit dosage form;
b) a therapeutically effective amount of one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents nd a pharmaceutically acceptable carrier in a second unit dosage form; and
c) a container for containing said first and second dosage forms.

66. A kit of claim 65 wherein said selective pyrazolopyrimidinone cGMP PDE5 inhibitor is sildenafil or sildenafil citrate.

67. A kit containing a treatment for type 2 diabetes mellitus comprising:
a) a therapeutically effective amount of a selective pyrazolopyrimidinone cGMP PDE5 inhibitor and a pharmaceutically acceptable carrier in a first unit dosage form;
b) a therapeutically effective amount of one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents nd a pharmaceutically acceptable carrier in a second unit dosage form; and
c) a container for containing said first and second dosage forms.

68. A kit of claim 67 wherein said selective pyrazolopyrimidinone cGMP PDE5 inhibitor is sildenafil or sildenafil citrate.

69. A kit containing a treatment for impaired glucose tolerance (IGT) comprising:
a) a therapeutically effective amount of a selective pyrazolopyrimidinone cGMP PDE5 inhibitor and a pharmaceutically acceptable carrier in a first unit dosage form;
b) a therapeutically effective amount of one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents nd a pharmaceutically acceptable carrier in a second unit dosage form; and
c) a container for containing said first and second dosage forms.

70. A kit of claim 69 wherein said selective pyrazolopyrimidinone cGMP PDE5 inhibitor is sildenafil or sildenafil citrate.

71. A kit containing a treatment for insulin resistance comprising:
a) a therapeutically effective amount of a selective pyrazolopyrimidinone cGMP PDE5 inhibitor and a pharmaceutically acceptable carrier in a first unit dosage form;
b) a therapeutically effective amount of one or more further components selected from one or more of: protein kinase inhibitors; and or one or more activators or AMP-activated protein kinase; and/or one or more weight loss agents; and/or insulin; and/or one or more PPAR-gamma agonists; and/or one or more PPAR-alpha agonists; and/or one or more dual PPAR-alpha/PPAR-gamma agonists; one or more sorbitol dehydrogenase inhibitors; one or more aldose reductase inhibitors; one or more insulin sensitising agents; and one or more hypoglycaemic agents nd a pharmaceutically acceptable carrier in a second unit dosage form; and
c) a container for containing said first and second dosage forms.

72. A kit of claim 71 wherein said selective pyrazolopyrimidinone cGMP PDE5 inhibitor is sildenafil or sildenafil citrate.
